(19) United States
(12) Patent Application Publication
(10) Pub. No.: US 2023/0137927 A1
(43) Pub. Date: May 4, 2023

(54) INTERFERON TAU AS ANTIVIRAL THERAPY

(71) Applicant: Southlake Pharmaceuticals, Inc., Cambridge, MA (US)

(72) Inventors: Yuhua George Zhang, Auburndale, MA (US); Wendy Wanjin Tang, College Station, TX (US)

(21) Appl. No.: 17/918,175

(22) PCT Filed: Apr. 22, 2021
(86) PCT No.: PCT/US21/28540

§ 371 Date: Oct. 11, 2022

Related U.S. Application Data
(60) Provisional application No. 63/029,002, filed on May 22, 2020.
(60) Provisional application No. 63/016,347, filed on Apr. 28, 2020.

(51) Int. Cl. A61K 38/21; A61K 45/06; A61P 31/14

(52) U.S. Cl. CPC A61K 38/21; A61K 45/06; A61P 31/14

(57) ABSTRACT

The invention generally relates to novel therapeutic uses of interferon tau as an antiviral agent. More particularly, the invention provides novel compositions of interferon tau and methods of therapeutic use thereof in treating viral infections and related diseases and conditions.

### PRIORITY CLAIMS AND RELATED PATENT APPLICATIONS

This application claims the benefit of priority to U.S. Provisional Application Ser. Nos. 63/016,347, filed Apr. 28, 2020, and 63/029,002, filed May 22, 2020, the entire content of each of which is incorporated herein by reference.

### TECHNICAL FIELD OF THE INVENTION

The invention generally relates to novel therapeutic uses of interferon tau as an antiviral agent. More particularly, the invention provides novel compositions of interferon tau and methods of therapeutic use thereof in treating viral (e.g., flavivirus and coronavirus) infections and related diseases and conditions.

### BACKGROUND OF THE INVENTION

Interferon tau (IFNT) is the pregnancy recognition signal secreted from trophectoderm of ruminant (cow, sheep, and goat) conceptuses (embryo and associated membranes). There is no functionally active human analog of IFNT. Ovine IFNT has been shown to have antiviral, anti-proliferative and immunomodulatory effects. (Bazer et al. 2010 Mol Hum Reprod 16(3): 135-152.)

IFNT is a member of type-I interferon (IFN) family. Within type I IFN family, it is most similar to IFN omega (IFNW) with about 70% amino acid (AA) identity. It has about 50% of AA identity with IFN alpha (IFNA) and about 25% AA identity with IFN beta (IFNB). Unlike IFNA, IFNB, and other Type I interferons, a striking feature of IFNT is that it does not have cytotoxicity even at high concentrations. (Soos et al. 1995 J Immunol 155(5): 2747-2753.) Ovine IFNT binds to type I IFN receptors on cells with high affinity, but less strongly than IFNA and IFNB, to induce comparable antiproliferative, antiviral and immunomodulatory activities, but without the known cytotoxicity of IFNA and IFNB. (Pontzer et al. 1991 Cancer Res 51(19): 5304-5307; Soos et al. 1995 J Immunol 155(5): 2747-2753; Bazer et al. 2010 Mol Hum Reprod 16(3): 135-152.)

Another unique property of IFNT is its oral availability, unlike most biologics. Oral administration of IFNT increases energy metabolism, reduces adiposity, and alleviates adipocytes inflammation and insulin resistance in rats and mice. (Tekwe et al. 2013 Biofactors 39(5): 552-563; Ying et al. 2014 PLoS One 9(6): e98835.) Human clinical studies have shown that thrice daily oral doses of 3 mg of IFNT for up to nine months was safe and well tolerated.

Flaviviruses include disease-causing viruses, such as Zika virus (ZIKV), Dengue virus (DENV), West Nile virus (WNV), Japanese Encephalitis virus (JEV), Yellow Fever Virus (YFV), and Powassan Virus. Most of these flaviviruses are transmitted by arthropod (mosquito or tick), which are classified as arboviruses. They belong to the family Flaviviridae and genus flavivirus. Viruses in this family have a single stranded, plus-sense viral RNA genome of approximately 11,000 nucleotides in length that encodes three structural (C, Env, M) and seven non-structural proteins (NS1, NS2A, NS2B, NS3, NS4A, NS4B, and NS5). (Lim 2019 Antiviral Res. 163:156-178.) The essential mechanism by which Flaviviruses penetrate human host cells is clathrin-mediated endocytosis, and then envelope conformation adjustment, membrane fusion and discharge of the viral genome. (Agrelli et al. 2019 Infect Genet Evol 69: 22-29.)

ZIKV was first detected in 1947 from a sentinel monkey in the Zika forest in Uganda, east Africa. Epidemiological research indicated that ZIKV was broadly distributed in sub-Saharan Africa and Southeast Asia. Since its emergence in Brazil in 2015, ZIKA has quickly spread throughout the Americas. Most ZIKV infections are subclinical or mild influenza-like illness, but severe infection has been found, such as Guillain-Barre syndrome in adults and microcephaly in babies vertically transmitted from infected mothers. ZIKV infection could be misdiagnosed as DENV virus infection because of clinical symptom and serological cross reactivity with closely related viruses. Neither an effective drug nor a vaccine treatment is available for ZIKV presently. Thus, the public health response mainly concentrates on prevention of the infection, especially in pregnant women. (Plourde et al. 2016 Emerg Infect Dis 22(7): 1185-1192.)

DENV also belongs to the family of Flaviviruses. DENV infects host cells by first binding to cell surface receptors, then entering the cell via a clathrin-dependent entry pathway. DENV-induced diseases are major causes of sickness and death in tropical and subtropical regions, where about 400 million people are infected annually. Dengue cases have increased four times during the last thirteen years, a much higher rate than other communicable diseases. (Wilder-Smith et al. 2019 Lancet 393(10169): 350-363.) There are four serotypes of DENV viruses (DENV1-4), co-circulating in over 140 countries. DENV infection is the tenth highest cause of both mortality and morbidity in developing countries and the leading cause of mortality in children under 15 years old in some South-East Asian countries. DENV presents a worldwide health problem because of enhanced territorial expansion of both DENV viruses and its vector, the Aedes Aegypti mosquitoes.

Currently there is no effective treatment of DENV infection, and little progress has been made for development of new therapy. (Wilder-Smith et al. 2019 Lancet 393(10169): 350-363.) Several antiviral drugs (e.g., chloroquine, balapiravir, celgosivir, and lovastatin) have been studied in clinical trials for DENV infection, but there is no clear evidence of benefit in lowering plasma viraemia or preventing infection-related complications. An alternative intervention involves corticosteroid therapy, which has demonstrated no convincing benefit on mortality from dengue shock syndrome. Since there is no effective antiviral or immunosuppressive therapeutic method, supportive care, such as judicious fluid resuscitation, is the first line treatment option of DENV infection, but none of these treatment methods is effective in preventing or treating clinically significant dengue-associated bleeding. (Wilder-Smith et al. 2019 Lancet 393(10169): 350-363.)

Another arthropod-borne flavivirus is Japanese Encephalitis virus (JEV). It is transmitted mostly by mosquito Culex tritaeniorhynchus and Culex vishnui. JEV is widely distributed in Asia, Western Pacific and Australia. Over three billion people are at a heightened risk for JEV infection. The case fatality rate is up to 30% among those with disease symptoms. The infection results in a spectrum of clinical illness that starts with flu-like symptoms, neck stiffness, disorientation, coma, seizures, spastic paralysis and finally death. JEV has caused serious public health problems not only due to a large number of deaths but also because of severe neuro-psychiatric sequelae that required lifelong support with socioeconomic burden. (Kulkarni et al. 2018 Open Virol J 12: 121-130.) Although the vaccines for Japanese Encephalitis vaccine is safe and effective, there is currently no effective treatment for JEV infection. About 68,000 new cases emerge each year globally. Safe and effective drugs are urgently needed to fight JEV. (Kulkarni et al. 2018 Open Virol J 12: 121-130.)

Yellow fever (YF) is another infectious disease caused by arthropod borne flavivirus, YFV. The symptoms of YFV infection includes influenza-like syndrome, severe liver and renal dysfunction, circulatory shock, and hemorrhage. After YF infection, Kupffer cells in the liver become infected on the first day. After that, the virus spreads to the kidney, bone marrow, spleen, and lymph nodes. The disease results in serious morbidity and mortality with fatality rates over 20%. Although an effective vaccine is available, YF remains a serious public health threat in sub-Saharan Africa and tropical South America, where YF causes regular epidemics with 200,000 cases and 30,000 deaths annually. There are no effective treatment options available for YFV infection. (Galbraith et al. 2009 Vaccines for Biodefense and Emerging and Neglected Diseases 753-785; Jentes et al. 2011 Lancet Infect Dis 11(8): 622-632.)

The COVID-19 outbreak, caused by severe acute respiratory syndrome coronavirus 2 (SARS-CoV-2), was declared by the World Health Organization as a Public Health Emergency of International Concern on Jan., 30, 2020 and as a pandemic on Mar. 11, 2020. As of April 2021, the COVID-19 pandemic rapidly grew to over 130 million cases across the globe, resulting in 3 million deaths, including over 550,000 in the United States. (COVID-19 Dashboard by the Center for Systems Science and Engineering (CSSE) at Johns Hopkins University. ArcGIS. Johns Hopkins University. Retrieved Apr. 13, 2021.)

In addition to escalating death tolls and sufferings across the globe, the COVID-19 pandemic has caused severe global social and economic disruptions, including skyrocketing unemployment, widespread supply shortages, and postponement or cancellation of educational, sporting, religious and cultural events. While vaccines effective against wild type COVID-19 are being rolled out, a number of variants have emerged and more are expected to surface. Containment and mitigation strategies so far have had limited impact in slowing down the spread of highly contagious and fast-moving variants.

Members of coronavirus family, of which COVID-19 is a member, are positive-sense single-stranded RNA virus genomes in the size ranging from 26 to 32 kilobases. They are enveloped and nonsegmented. They have the largest known viral RNA genome. The virion has a nucleocapsid, which consists of genomic RNA and phosphorylated nucleocapsid (N) protein. N protein is contained inside phospholipid bilayers and wrapped by two different types of spike proteins: the spike glycoprotein trimmer (S) possessed by all CoVs, and the hemagglutinin-esterase (HE) that is present in a few CoVs. There are also membrane (M) protein (a type III transmembrane glycoprotein) and the envelope (E) protein next to the S proteins in the virus envelope. (Li, et al. 2020 J Med Virol 92(4): 424-432; Sternberg, et al. 2020 Life Sci 257: 118056.)

There are four genera in the coronavirus family Coronaviridae, i.e., α, β, γ, and δ coronaviruses. 30 CoVs are found to infect humans, mammals, fowl, and other animals. α- and β-CoVs cause human infections. CoVs are common human pathogens. Human Coronavirus 229E (hCoV-229E) is an α-CoV responsible for common cold. SARS (severe acute respiratory syndrome CoV) related viruses (including COVID-19 virus/SARS-CoV-2) and MFRS (Middle East respiratory syndrome CoV) related viruses, and another common cold virus OC43 are β-CoVs. They all belong to the same coronavirus family Coronavirividae. These viruses cause severe pneumonia, dyspnea, renal insufficiency, and even death possibly due to over-reacted immune response. (Li, et al. 2020 J Med Virol 92(4): 424-432; Chan, et al. 2015 Clin Microbiol Rev 28(2): 465-522; Cheng, et al. 2007 Clin Microbiol Rev 20(4): 660-694; Zumla, et al. 2016 Nat Rev Drug Discov 15(5): 327-347; Woo, et al. 2009 Exp Biol Med (Maywood) 234(10): 1117-1127; Khan, et al. 2020 J Clin Microbiol 58(5); Cui, et al. 2019 Nat Rev Microbiol 17(3): 181-192; Fung, et al. 2019 Annu Rev Microbiol 73: 529-557.)

Human Coronavirus 229E (hCoV-229E) is an α-CoV. SARS (severe acute respiratory syndrome CoV) related viruses (including COVID-19 virus/SARS-CoV-2) and MERS (Middle East respiratory syndrome CoV) related viruses are β-CoVs (Zumla, Chan et al. 2016). They all belong to the same coronavirus family Coronavirividae. These viruses cause severe pneumonia, dyspnea, renal insufficiency, and even death possibly due to over-reacted immune response. (Li, et al. 2020 J Med Virol 92(4): 424-432; Cheng, et al. 2007 Clin Microbiol Rev 20(4): 660-694.) These viruses are related with severe epidemic human diseases with high morbidity and mortality rates. COVID-19 is already declared as a global pandemic.

Human interferons were reported to inhibit SARS in vitro. (Cinatl, et al. 2003 Lancet 362(9380): 293-294.) However, due to their serious side effects, they are not ideal therapeutic candidates for SARS treatment. Because our newly generated PEGylated IFNT and the original IFNT have low cell toxicity, and potent anti-CoV229E activity, they are very promising potential therapeutic candidates to treat COVID-19.

Various antiviral medications are under investigation for COVID-19, as well as medications targeting the immune response; however, none has yet been scientifically established to be clearly effective on mortality in published randomised controlled trials. (See, e.g., Sanders, et al. (April 2020) “Pharmacologic Treatments for Coronavirus Disease 2019 (COVID-19): A Review” JAMA 323 (18): 1824-1836; Mulaw 2020 Clin Pharmacol 12: 203-212; Stasi, et al. 2020 Eur J Pharmacol 889: 173644; Panati, et al. 2020 “An overview on COVID-19 pandemic: from discovery to treatment.” Infect Disord Drug Targets; Ghaffari, et al. 2021 Emergent Mater: 1-16.) As of now, remdesivir and some monoclonal antibodies are the only agents that may have an effect on the time it takes to recover from the virus. (“NIH Clinical Trial Shows Remdesivir Accelerates Recovery from Advanced COVID-19”. National Institute of Allergy and Infectious Diseases. Retrieved 2 May 2020; “COVID19 treatment guidelines” (NIAID.NIH.GOV 2021) An interim authorisation for remdesivir and some monoclonal antibodies were granted by the US FDA under emergency use for people hospitalised with severe COVID-19. Several agents, such as hydroxychloroquine or chloroquine which were previously thought of or proclaimed to be effective, have since been shown to have little effect or may even be harmful.

In sum, flavivirus and coronavirus infections and related diseases continue to pose serious public health concerns. Drugs with improved efficacy and safety remain in urgent demand.

### BRIEF DESCRIPTION OF THE DRAWINGS

FIG. 1 and TABLE 1 show exemplary data on the inhibitory activity of IFNT against ZIKV in plaque reduction assay. IFNT inhibited ZIKV in plaque reduction assay in a dose-dependent manner. EC50 (50 percent effective inhibitory concentration) of IFNT against ZIKV is less than 20 ng/mL (1 nM). IFN-β was used as a positive control and had an EC50 of 4.66 IU/mL in this assay.

FIG. 2 and TABLE 2 show exemplary data on IFNT inhibition of ZIKV in cytopathic effects (CPE) assay by pre-incubation for either overnight or 1 hr. IFNT inhibited ZIKAV in this assay in a dose-dependent manner. EC50 of IFNT is 27.8 ng/mL (1.40 nM). IFN-β was used as positive control and had an EC50 of 5.39 IU/mL in this assay. 50% of cytotoxicity concentration (CC50) of IFNT was assessed in parallel with the antiviral activity. CC50 was greater than the highest concentration of IFNT used in the assay (>300 ng/mL).

FIG. 3 and TABLE 3 show exemplary data on the inhibitory activity of IFNT against DENV in plaque reduction assay. IFNT inhibited DENV in plaque reduction assay in a dose-dependent manner. The EC50 of IFNT against DENV virus is 1.1 ng/mL (0.05 nM). EC50 of the positive control Ribavirin against DENV virus is 32.20 μM.

FIG. 4A and TABLE 4 show exemplary data on the inhibitory activity of IFNT against YFV and JEV viruses in plaque reduction assay. IFNT inhibited YFV and JEV in a dose-dependent manner with an EC50 of 69 ng/mL (3.47 nM) for YFV and 4.6 ng/mL (0.2292 nM) for JEV. CC50 was assessed in parallel with the antiviral activity and was greater than the highest concentration of IFNT used in both assays (>600 ng/mL). FIG. 4B shows exemplary data on the inhibitory activity of Ribavirin against YFV and JEV viruses in plaque reduction assay. EC50 of Ribavirin for JEV is 35.2 μM. EC50 of Ribavirin for YFV is 136 μM.

FIG. 5A. shows exemplary anti-SARS-CoV-2 activity of IFNT.

FIG. 5B. shows exemplary cell viability assay of IFNT.

FIG. 5C. shows exemplary anti-SARS-CoV-2 activity of the reference compounds: remdesivir, chloroquine, hydroxychloroquine, aloxistatin, calpain inhibitor IV.

FIG. 6. shows exemplary dose-response curves of IFNT in inhibiting hCoV OC43 in CPE and cell viability assay.

FIG. 7 shows exemplary CPE and cell viability data of in vitro anti-hCoV-229E activity of IFNT and remdesivir.

FIG. 8. Shows the two protein sequences of IFNT.

### SUMMARY OF THE INVENTION

The invention is based in part on the unexpected discovery of IFNT-based anti-infective therapeutics and methods of treatment and use thereof. This invention provides a broad therapeutic potential of using IFNT to treat human flavivirus and coronavirus infections with improved safety partly due to several unique therapeutic advantages of IFNT as compared to other Type I interferons, such as oral administration, pregnancy-friendly and minimum cytotoxicity. For example, IFNT (e.g., recombinant ovine IFNT) can be used to effectively inhibit viral replication of flaviviruses and coronaviruses including ZIKA, DENV, YFV, JEV, HCoV-229E and SARS-CoV-2 in human cells.

In one aspect, the invention generally relates to a method for treating a viral infection, or a related disease or condition. The method includes administering to a subject in need thereof a therapeutically effective amount of interferon tau (IFNT) and a pharmaceutically acceptable excipient, carrier, or diluent. The viral infection comprises one or more infections of flavivirus.

In another aspect, the invention generally relates to a method for inhibiting viral replication in cells. The method includes administering to a subject in need thereof an amount of IFNT effective to inhibit viral replication in the cells, wherein the inhibited virus is selected from flavivirus.

In yet another aspect, the invention generally relates to a pharmaceutical composition. The pharmaceutical composition includes IFNT and a pharmaceutically acceptable excipient, carrier, or diluent.

In yet another aspect, the invention generally relates to a unit dosage form having a pharmaceutical composition disclosed herein.

In yet another aspect, the invention generally relates to use of IFNT for treating a viral infection, wherein the viral infection comprises one or more infections of flavivirus.

In yet another aspect, the invention generally relates to use of IFNT in preparation of a medicament effective for treating a viral infection, wherein the viral infection comprises infections of flavivirus or coronavirus.

### Definitions

Unless defined otherwise, all technical and scientific terms used herein have the same meaning as commonly understood by one of ordinary skill in the art to which this invention belongs. The following terms, unless indicated otherwise according to the context wherein the terms are found, are intended to have the following meanings.

As used herein, the term “cell” refers to any prokaryotic, eukaryotic, primary cell or immortalized cell line, any group of such cells as in, a tissue or an organ. Preferably the cells are of mammalian (e.g., human) origin and can be infected by one or more pathogens.

As used herein, the terms “disease” or “disorder” refer to a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the term “host cell” refers to an individual cell or a cell culture that can be or has been a recipient of any recombinant vector(s) or isolated polynucleotide(s). A host cell can be a transfected, transformed, transduced or infected cell of any origin, including prokaryotic, eukaryotic, mammalian, avian, insect, plant or bacteria cells, or it can be a cells of any origin that can be used to propagate a nucleic acid described herein. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell that comprises a recombinant vector of the invention may be called a “recombinant host cell.”

Host cells include, without limitation, the cells of mammals, plants, insects, fungi and bacteria. Bacterial cells include, without limitation, the cells of Gram-positive bacteria such as species of the genus Bacillus, Streptomyces and Staphylococcus and cells of Gram-negative bacteria such as cells of the genus Escherichia and Pseudomonas. Fungal cells include, preferably, yeast cells such as Saccharomyces, Pichia pastoris and Hansenula polymorpha. Insect cells include, without limitation, cells of Drosophila and Sf9 cells. Plant cells include, among others, cells from crop plants such as cereals, medicinal or ornamental plants or bulbs. Suitable mammal cells for the present invention include epithelial cell lines (porcine, etc.), osteosarcoma cell lines (human, etc.), neuroblastoma cell lines (human, etc.), epithelial carcinomas (human, etc.), glial cells (murine, etc.), liver cell lines (monkey, etc.). CHO cells (Chinese Hamster Ovary), COS cells, BHK cells, cells HeLa, 911, AT1080, A549, 293 or PER.C6, human ECCs NTERA-2 cells, D3 cells of the line of mESCs, human embryonic stem cells such as HS293 and BGV01, SHEF1, SHEF2 and HS181, cells NIH3T3, 293T, REH and MCF-7 and hMSCs cells.

As used herein, the term “high dosage” is meant at least 5% (e.g., at least 10%, 20%, 50%, 100%, 200%, or even 300%) more than the highest standard recommended dosage of a particular compound for treatment of any human disease or condition.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., of a IL15 or IL15Rα sequence), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A preferred example of algorithm that is suitable for determining percent sequence identity and sequence similarity are the BLAST algorithms, which are described in Altschul et al. 1977 Nuc. Acids Res. 25:3389-3402 and Altschul et al. 1990 J Mol. Biol. 215:403-410, respectively. BLAST software is publicly available through the National Center for Biotechnology Information on the worldwide web at ncbi.nlm.nih.gov/. Both default parameters or other non-default parameters can be used. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

As used herein, the term “inhibit” refers to any measurable reduction of biological activity. Thus, as used herein, “inhibit” or “inhibition” may be referred to as a percentage of a normal level of activity.

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “polynucleotide,” “nucleic acid molecule,” “nucleotide,” “oligonucleotide,” and “nucleic acid” are used interchangeably herein to refer to polymeric forms of nucleotides, including ribonucleotides as well as deoxyribonucleotides, of any length. They can include both double-, single-stranded or triple helical sequences and include, but are not limited to, cDNA from viral, prokaryotic, and eukaryotic sources; mRNA; genomic DNA sequences from viral (e.g., DNA viruses and retroviruses) or prokaryotic sources; RNAi; cRNA; antisense molecules; recombinant polynucleotides; ribozymes; and synthetic DNA sequences. The term also captures sequences that include any of the known base analogs of DNA and RNA. Nucleotides can be referred to by their commonly accepted single-letter codes.

Polynucleotides are not limited to polynucleotides as they appear in nature, and also include polynucleotides where unnatural nucleotide analogues and inter-nucleotide bonds appear. A nucleic acid molecule may comprise modified nucleic acid molecules (e.g., modified bases, sugars, and/or internucleotide linkers). Non-limitative examples of this type of unnatural structures include polynucleotides wherein the sugar is different from ribose, polynucleotides wherein the phosphodiester bonds 3′-5′ and 2′-5′ appear, polynucleotides wherein inverted bonds (3′-3′ and 5′-5′) appear and branched structures. Also, the polynucleotides of the invention include unnatural inter-nucleotide bonds such as peptide nucleic acids (PNA), locked nucleic acids (LNA), C1-C4 alkylphosphonate bonds of the methylphosphonate, phosphoramidate, C1-C6 alkylphosphotriester, phosphorothioate and phosphorodithioate type. In any case, the polynucleotides of the invention maintain the capacity to hybridize with target nucleic acids in a similar way to natural polynucleotides.

Unless otherwise indicated or obvious from context, a particular nucleic acid sequence also implicitly encompasses conservatively modified variants thereof (e.g., degenerate codon substitutions) and complementary sequences, as well as the sequence explicitly indicated. Degenerate codon substitutions can be achieved by generating sequences in which the third position of one or more selected (or all) codons is substituted with mixed-base and/or deoxyinosine residues. (Batzer et al. 1991 Nucleic Acid Res. 19:5081; Ohtsuka et al. 1985 J. Biol. Chem. 260:2605-2608; Rossolini et al. 1994 Mol. Cell. Probes 8:91-98.)

As used herein, the terms “protein” and “polypeptide” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, a polypeptide may refer to a protein which includes modifications, such as deletions, additions, and substitutions (generally conservative in nature), to the native sequence, as long as the protein maintains the desired activity. These modifications may be deliberate or may be accidental. Amino acids can be referred to herein by either their commonly known three letter symbols or by the one-letter symbols recommended by the IUPAC-IUB Biochemical Nomenclature Commission.

As used herein, the term “purified” refers to a protein that may be substantially or essentially free of components that normally accompany or interact with the protein as found in its naturally occurring environment, i.e. a native cell, or host cell in the case of a recombinantly produced protein. A protein that may be substantially free of cellular material includes preparations of protein having less than about 30%, less than about 20%, less than about 15%, less than about 10%, less than about 5%, less than about 4%, less than about 3%, less than about 2%, or less than about 1% (by dry weight) of contaminating protein(s). When a protein or variant thereof is recombinantly produced by the host cells, the protein may be present at about 30%, at about 20%, about 15%, about 10%, about 5%, about 4%, about 3%, about 2%, or about 1% or less of the dry weight of the cells. When a protein or variant thereof is recombinantly produced by the host cells, the protein may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. Thus, a “substantially purified” protein may have a purity level of at least about 80%, specifically, a purity level of at least about 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Proteins and prodrugs of the present invention are, subsequent to their preparation, preferably isolated and/or purified to obtain a composition containing an amount by weight equal to or greater than 80% (“substantially pure”), which is then used or formulated as described herein. In certain embodiments, the compounds of the present invention are more than 95% pure.

As used herein, the term “recombinant,” with respect to a nucleic acid molecule, means a polynucleotide of genomic, cDNA, viral, semisynthetic, and/or synthetic origin which, by virtue of its origin or manipulation, is not associated with all or a portion of the polynucleotide with which it is associated in nature. The term “recombinant”, as used with respect to a protein or polypeptide, means a polypeptide produced by expression of a recombinant polynucleotide. The term “recombinant” as used with respect to a host cell means a host cell into which a recombinant polynucleotide has been introduced.

As used herein, the term “recombinant virus” refers to a virus that is genetically modified by the hand of man. The phrase covers any virus known in the art.

As used herein, the term “sample” refers to a sample from a human, animal, or to a research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

As used herein, the terms “subject” and “patient” are used interchangeably herein to refer to a living animal (human or non-human). The subject may be a mammal. The terms “mammal” or “mammalian” refer to any animal within the taxonomic classification mammalia. A mammal may be a human or a non-human mammal, for example, dogs, cats, pigs, cows, sheep, goats, horses, rats, and mice. The term “subject” does not preclude individuals that are entirely normal with respect to a disease or condition, or normal in all respects.

As used herein, the term “therapeutically effective amount” refers to the dose of a therapeutic agent or agents sufficient to achieve the intended therapeutic effect with minimal or no undesirable side effects. A therapeutically effective amount can be readily determined by a skilled physician, e.g., by first administering a low dose of the pharmacological agent(s) and then incrementally increasing the dose until the desired therapeutic effect is achieved with minimal or no undesirable side effects.

As used herein, the terms “treatment” or “treating” a disease or disorder refers to a method of reducing, delaying or ameliorating such a condition, or one or more symptoms of such disease or condition, before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “at least” a specific value is understood to be that value and all values greater than that value.

As used herein, “more than one” is understood as 2, 3, 4, 5, 6, 7, 8, 9, 10, 11, 12, 13, 14, 15, 16, 17, 18, 19, 20, 25, 30, 40, 50, 100, etc., or any value therebetween.

Unless specifically stated or obvious from context, as used herein, the term “or” is understood to be inclusive.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Any compositions or methods disclosed herein can be combined with one or more of any of the other compositions and methods provided herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### DETAILED DESCRIPTION OF THE INVENTION

The invention provides IFNT-based anti-infective therapeutics and methods of treatment and use thereof. The anti-flavivirus and anti-coronavirus activities of IFNT for ZIKAV, DENV, YFV, JEV, HCoV229E and SARS-CoV-2 viruses are disclosed herein. This invention provides a broad therapeutic potential of using IFNT to treat human flavivirus and coronavirus infections with improved safety partly due to several unique therapeutic advantages of IFNT as compared to other Type I interferons, such as oral administration, pregnancy-friendly and minimum cytotoxicity. For example, IFNT (e.g., recombinant ovine IFNT) can be used to effectively inhibit viral replication of flaviviruses including ZIKA, DENV, YFV and JEV in human cells.

In one aspect, the invention generally relates to a method for treating a viral infection, or a related disease or condition. The method includes administering to a subject in need thereof a therapeutically effective amount of interferon tau (IFNT) and a pharmaceutically acceptable excipient, carrier, or diluent. The viral infection comprises one or more infections of flavivirus.

In another aspect, the invention generally relates to a method for inhibiting viral replication in cells. The method includes administering to a subject in need thereof an amount of IFNT effective to inhibit viral replication in the cells, wherein the inhibited virus is selected from flavivirus.

In certain embodiments, the viral infection includes infection of a flavivirus. In certain embodiments, the viral infection includes coronavirus infection.

In certain embodiments, the viral infection includes infection of one or more of ZIKA, DENV, YFV, JEV, West Nile, Powassan, HcoV-229E and SARS-CoV-2 viruses.

In certain embodiments, the viral infection comprises infection of a SARS-related coronavirus. In certain embodiments, the viral infection comprises infection of SARS-CoV-2. In certain embodiments, the viral infection comprises infection of one or more variants of SARS-CoV-2, e.g., B.1.1.7, B.1.351, P.1, B.1.427, or B.1.429 variants (http://www.edc.gov/coronavirus/2019-ncov/cases-updates/variant-surveillance/variant-infi.html accessed on Apr. 14, 2021).

A number of diseases and conditions related to COVID-19 and common cold may be treated or reduced using the method of the invention.

In certain embodiments, the related disease or condition is pneumonia.

In certain embodiments, the related disease or condition is ARDS.

In certain embodiments, the related disease or condition is an inflammatory disorder.

In certain embodiments, the related disease or condition is a cardiovascular disorder.

In certain embodiments, the related disease or condition is a common cold.

In certain embodiments, the related disease or condition is flu.

In certain embodiments, the IFNT is mammalian IFNT. In certain embodiments, the IFNT is non-human mammalian IFNT. In certain embodiments, the mammalian IFNT is that of ovine or bovine IFNT. In certain embodiments, the IFNT is ovine IFNT. In certain embodiments, the IFNT is recombinant ovine IFNT.

In certain embodiments, the IFNT comprises an amino acid sequence that is at least 70% (e.g., at least 80%, at least 90%, at least 95%, at least 99%) homologous with SEQ ID No. 1 or SEQ ID No. 2.

In certain embodiments, the IFNT comprises an amino acid sequence set forth in SEQ ID NO. 1.

In certain embodiments, the IFNT comprises an amino acid sequence set forth in SEQ ID NO. 2.

In certain embodiments of the method, the administration includes oral administration.

In certain embodiments, the administration comprises intravenous administration.

In certain embodiments, IFNT is administered at a dosage in the range from about 0.1 mg to about 200 mg (e.g., from about 0.1 mg to about 150 mg, from about 0.1 mg to about 100 mg, from about 0.1 mg to about 50 mg, from about 0.1 mg to about 10 mg, from about 0.1 mg to about 1 mg, from about 1 mg to about 200 mg, from about 10 mg to about 200 mg, from about 50 mg to about 200 mg, from about 100 mg to about 200 mg) per day.

In certain embodiments, the method includes administering to the subject a second therapeutic agent. The second therapeutic agent may be any suitable therapeutic agent, for example, a second antiviral agent. In certain embodiments, the second antiviral agent is nucleotide or nucleoside analog antiviral agent.

In certain embodiments, the second antiviral agent is a nucleos(t)ide inhibitor. In certain embodiments, the second antiviral agent is selected from the group consisting of chloroquine, balapiravir, celgosivir, lovastatin, ribavirin, simeprevir and sofosbuvir.

In certain embodiments, the second antiviral agent is a type I or type II interferon. In certain embodiments, the second antiviral agent is selected from the group consisting of peginterferon alfa-2b or peginterferon alfa-2a and peginterferon beta-1.

The second therapeutic agent may be administered prior to, concomitant with or after the administration of IFNT.

In yet another aspect, the invention generally relates to a pharmaceutical composition. The pharmaceutical composition includes IFNT and a pharmaceutically acceptable excipient, carrier, or diluent.

In certain embodiments, the pharmaceutical composition further includes a second therapeutic agent. The second therapeutic agent may be any suitable therapeutic agent, for example, a second antiviral agent.

In certain embodiments, the second antiviral agent is a nucleos(t)ide inhibitor, e.g., selected from the group consisting of chloroquine, balapiravir, celgosivir, lovastatin, ribavirin, simeprevir and sofosbuvir,

In certain embodiments, the second antiviral agent can also be a protease inhibitor, e.g., selected from the group consisting of saquinavir , ritonavir, indinavir, nelfinavir, lopinavir-ritonavir, atazanavir, fosamprenavir, tipranavir, darunavir, darunavir plus cobicistat, simeprevir, asunaprevir and vaniprevir.

In certain embodiments, the second antiviral agent is a type I or type II interferon

In certain embodiments, the second antiviral agent is selected from the group consisting of interferon alfa-2a (Roferon-A), interferon alfa-2b (intron-A), interferon alfa-n3 (Alferon-N), peginterferon alfa-2b (PegIntron Sylatron), interferon beta-1a (Avonex), interferon beta-1a (Rebif), interferon beta-1b (Betaseron), interferon beta-1b (Extavia), interferon gamma-1b (Actimmune), peginterferon alfa-2a (Pegasys ProClick), peginterferon alfa-2a and ribavirin (Peginterferon), peginterferon alfa-2b and ribavirin (Peglntron/Rebetol Combo Pack), peginterferon beta-1a (Plegridy), and interferon alfacon-1. In certain embodiments, the second antiviral agent is peginterferon alfa-2b, peginterferon alfa-2a or peginterferon beta-1.

In certain embodiments, the pharmaceutical composition is suitable for oral administration to a subject suffering from a viral infection, e.g., one or more infections of flavivirus.

In certain embodiments, the pharmaceutical composition is suitable for intravenous administration to a subject suffering from a viral infection, e.g., one or more infections of flavivirus.

In certain embodiments, the pharmaceutical composition is suitable for intramuscular and/or subcutaneous administration.

In certain embodiments, the pharmaceutical composition is suitable for inhaled administration.

In yet another aspect, the invention generally relates to a unit dosage form having a pharmaceutical composition disclosed herein.

In yet another aspect, the invention generally relates to use of IFNT for treating a viral infection, wherein the viral infection comprises one or more infections of flavivirus.

In yet another aspect, the invention generally relates to use of IFNT in preparation of a medicament effective for treating a viral infection, wherein the viral infection comprises one or more infections of flavivirus.

In certain embodiments, use of IFNT is for treating an infection of a flavivirus.

In certain embodiments, use of IFNT is for treating an infection of one or more of ZIKA, DENV, YFV, JEV, West Nile and Powassan viruses.

In certain embodiments, use of IFNT includes using a mammalian IFNT.

In certain embodiments, use of IFNT includes using a non-human mammalian IFNT.

In certain embodiments, use of IFNT includes using a recombinant IFNT.

In certain embodiments, the IFNT comprises an amino acid sequence that is at least 70% (e.g., at least 75%, at least 80%, at least 85%, at least 90%, at least 95%, at least 99%) homologous with SEQ ID No. 1 or SEQ ID No. 2.

In certain embodiments, the IFNT comprises an amino acid sequence set forth in SEQ ID NO. 1.

In certain embodiments, the IFNT comprises an amino acid sequence set forth in SEQ ID NO. 2.

### EXAMPLES

The Examples below describe certain exemplary embodiments of compounds prepared according to the disclosed invention. It will be appreciated that the following general methods, and other methods known to one of ordinary skill in the art, can be applied to compounds and subclasses and species thereof, as disclosed herein.

### Example 1

IFNT's anti-ZIKA virus property was evaluated in vitro in plaque reduction and cytopathic effect (CPE) assays.

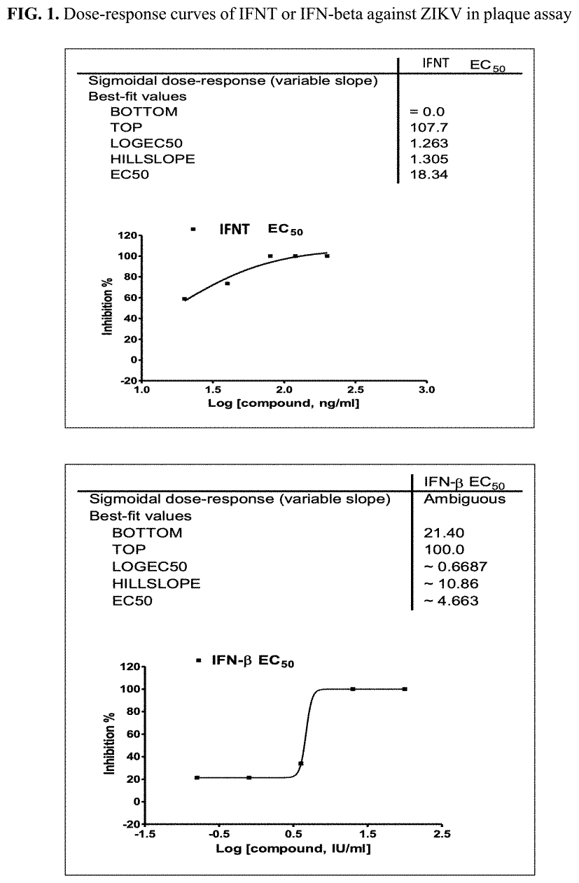

(A) The experimental procedure for the plaque reduction assay for TABLE 1 and FIG. 1 is as follows: The cells used in the assay were VERO cells obtained from ATCC, which were kidney epithelial cells from an African green monkey Cercopithecus aethiops (ATCC #CCL-81). The IFNT was a recombinantly produced protein from yeast. (Van Heeke et al. 1996 J Interferon Cytokine Res 16(2): 119-126.)

VERO cells were seeded at 600,000 cells/well in the 6-well plates and grown at 37° C. and 5% CO₂ for 4-6 hr. The supernatants of assay plate were replaced with assay medium containing 1×final test concentrations of IFNT. Cells were grown at 37° C. and 5% CO₂ overnight. Next day, each well was replenished with 0.5 mL/well of assay medium containing 2×final test concentrations of IFNT. Cells were infected with 0.5 mL/well, 40-60 PFU/well virus. The inocula was incubated at 37° C. and 5% CO₂ for 2 hr. Then the medium was replaced with the low melting point agarose medium containing 1×final test concentrations of IFNT. Cells were cultured at 37° C. and 5% CO₂ for 4 days and the plaques were dyed with crystal violet. IFN-β was used as the positive control of the assay and was treated with the identical procedure as IFNT except that the pre-incubation time was 1 hr instead of overnight. The antiviral activity of the compounds was calculated based on the reduction of plaque numbers at each concentration normalized by the virus control. EC₅₀ values were calculated with GraphPad Prism software.

The compound antiviral activity (% Inhibition) was calculated using the equation below:

% inhibition=100−100*(plaque No of sample treated well/plaque No of virus control well)

Dose-response curves were plotted and the EC₅₀ values were calculated by using the GraphPad Prism software.

TABLE 1 and FIG. 1 show the evaluation of inhibitory activity of IFNT against ZIKV in plaque reduction assay. Both IFNT and reference IFN-β were tested at 5 concentrations. ZIKV strain used for cell infection was PRVABC59 (ATCC#VR-1843).

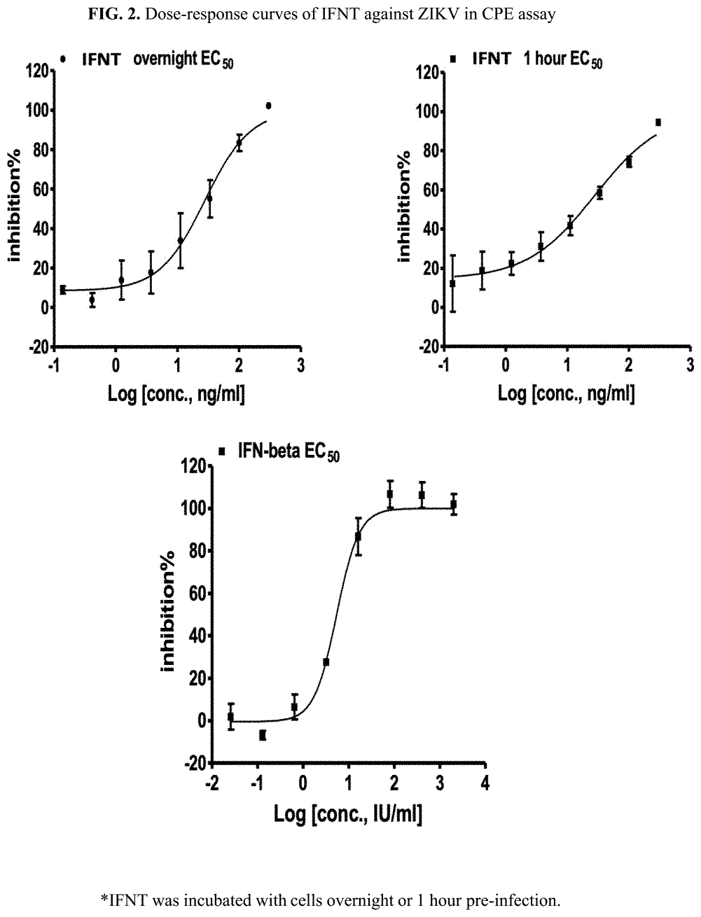

(B) Zika CPE assay procedure for TABLE 2 and FIG. 2 is as follows: Huh-7 cells were seeded at a density of 10,000 cells/well in microwell plates, and cultured at 37° C. and 5% CO₂ overnight. Next day, cells were replenished with medium containing appropriate concentrations of test compounds for overnight or 1 hr incubation before virus infection. MOI is 0.04 to yield 80-95% CPE. The resulting cultures were kept under the same conditions for additional 3 days until virus infection in virus control displays significant CPE. Cell viability was measured with CCK8 or CellTiter Glo following the manufacturer's instruction. Antiviral activity was calculated based on the inhibition of virus-induced CPE at each concentration normalized by the mock control. EC₅₀ values were calculated with GraphPad Prism software. Cytotoxicity of test compounds were assessed under the same conditions, but without virus infection, in parallel. Data were used to calculate % viability of Huh-7 cells. CC₅₀ values were calculated based on inhibition of cell proliferation using the GraphPad Prism software.

TABLE 2 and FIG. 2 demonstrate that ovine IFNT potently reduces ZIKA virus infection with an EC50 of 27.9 ng/mL (1.40 nM) in CPE assay. Overnight pre-incubation or lhr preincubation with IFNT before viral infection achieved similar inhibitory effects and comparable EC50s.

### Example 2

IFNT was evaluated in vitro for its anti-DENV activity in plaque reduction assay.

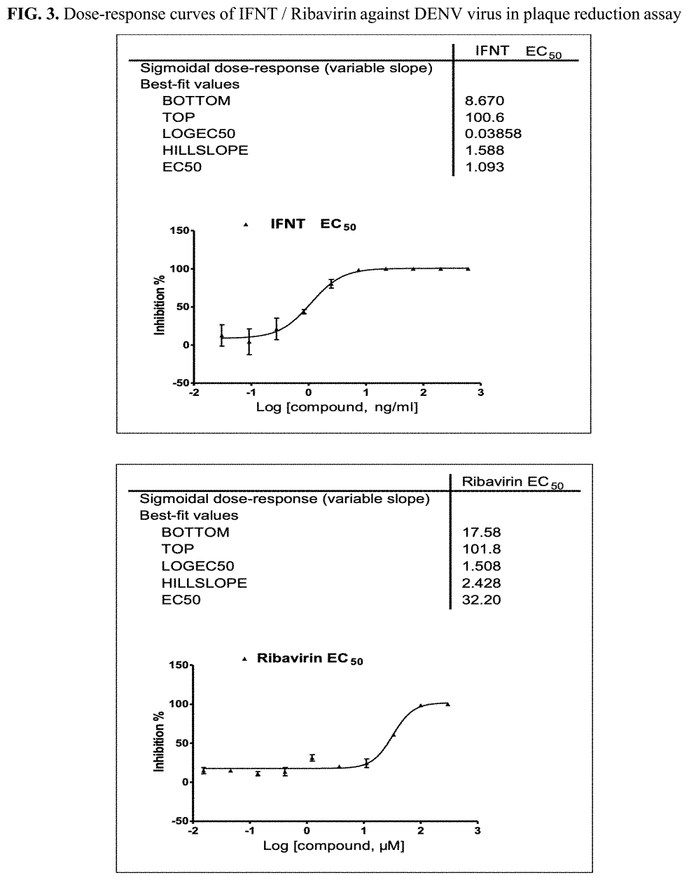

The plaque reduction assay experimental procedure for TABLE 3 and FIG. 3 is as follows: The strain of DENV virus used in the assay is type 2 Rluc-NGC strain (Institute Pasteur of Shanghai, CAS). Vero cells are from ATCC #CCL-81. Ribavirin, a nucleoside antiviral compound, was used as the reference compound in this study.

VERO cells were seeded at 600,000 cells/well in 6-well plates and cultured at 37° C. and 5% CO₂ for 4-6 hr. Then the supernatants of assay plate were replaced with assay medium containing 1×final test concentrations of IFNT. Cells were incubated at 37° C. and 5% CO₂ overnight. Next day, each well was replenished with 0.5 mL/well of assay medium containing 2×final test concentrations of IFNT. Ribavirin was used as the positive control of the assay, and was treated with the identical procedure as IFNT except the pre-incubation time was 1 hr instead of overnight. Cells were infected with 0.5 ml/well, 40-60 PFU/well virus. The inocula was incubated at 37° C. and 5% CO₂ for 2 hr. Then the medium was replaced with the low melting point agarose medium containing 1×final test concentrations of IFNT or ribavirin. Cells were cultured at 37° C. and 5% CO₂ for 7 days and the plaques were dyed with crystal violet.

TABLE 3 and FIG. 3 show that IFNT suppressed DENV infection potently with an EC₅₀ of 1.09 ng/mL (0.05 nM), whereas Ribavirin has an EC₅₀ of 32.2 μM in this assay. IFNT is 644,000 times more potent than Ribavirin in this assay.

### Examples 3 & 4

IFNT was evaluated in plaque reduction assay for its anti-YFV and anti-JEV properties.

The procedure of plaque reduction assay of IFNT against YFV and JEV is as follows:

The strain of JEV (JEV) used in the assay is 14-14-2. The strain of YFV used is 17D.

Ten 3-fold serial dilutions of IFNT starting at 600 ng/mL were incubated with cells seeded in 24-well culture plates in duplicate overnight. Virus was added to cells at 400 plaque forming units (PFUs) and incubated for one hr. A 0.8% methylcellulose overlay containing IFNT dilutions at 1×final concentration was added to corresponding cells. Cells were incubated for seven days for YFV and ten days for JEV at 37° C. in a humidified 5% CO₂ atmosphere. Cells were fixed and stained with crystal violet. Viral plaque counts were used to calculate EC₅₀ using XLfit dose response model.

### Cytotoxicity Assay of IFNT was Performed as Follows:

Ten 3-fold serial dilutions of IFNT starting at 600 ng/mL were incubated with cells seeded in 96-well culture plates in triplicate overnight. Incubation medium containing IFNT dilutions at 1×final was added to corresponding cells. Cells were incubated for ten days. Cell viability was determined using CellTiter-Glo kit. CC₅₀ was calculated using XLfit dose response model.

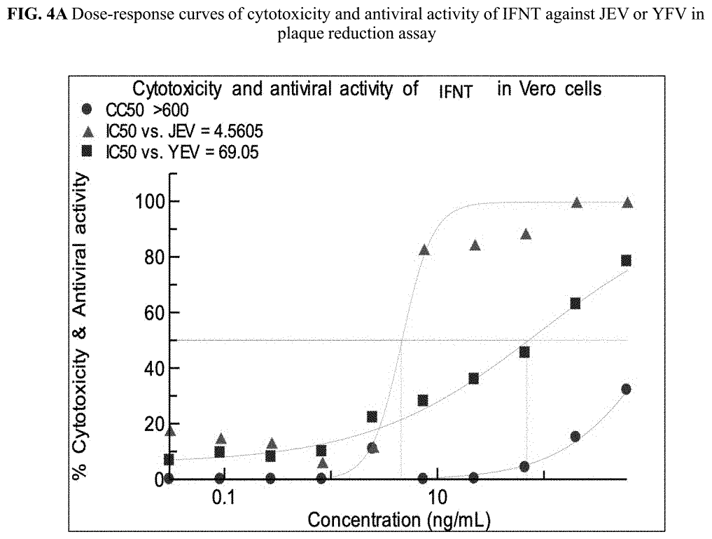

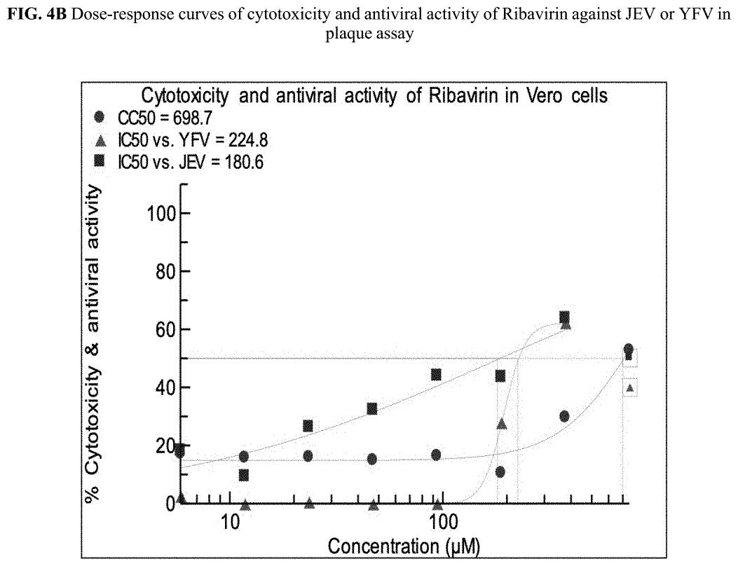

TABLE 4 and FIG. 4A show that IFNT inhibited YFV and JEV viruses in plaque reduction assay in a dose-dependent manner. The EC₅₀ of IFNT for JEV was 4.6 ng/mL (0.23 nM). EC50 of IFNT for YFV was 69 ng/mL (3.47 nM). FIG. 4A also shows the dose response curve of IFNT in cytotoxicity assay. It indicates that CC₅₀ of IFNT was greater than 600 ng/mL (30.15 nM), the highest concentration used in the assay.

FIG. 4B shows a dose response curve of Ribavirin in this assay. It shows that the EC₅₀ of Ribavirin for JEV is 180.6 μM/mL. EC₅₀ of Ribavirin for YFV is 224.8 μM/mL. IFNT was found to be 64,000 times more potent than Ribavirin, a marketed antiviral drug for YFV and 785,000 times more potent than Ribavirin for JEV. CC₅₀ of Ribavirin was more than 698.7 μM/mL.

### Example 5

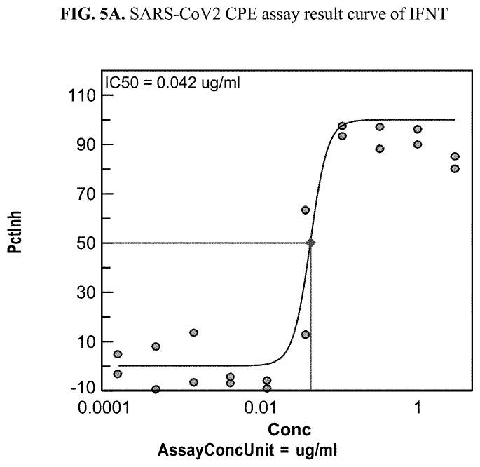

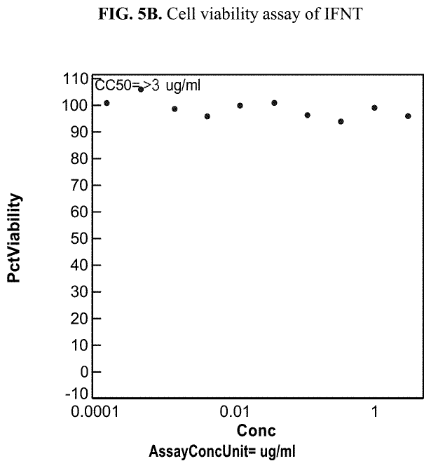

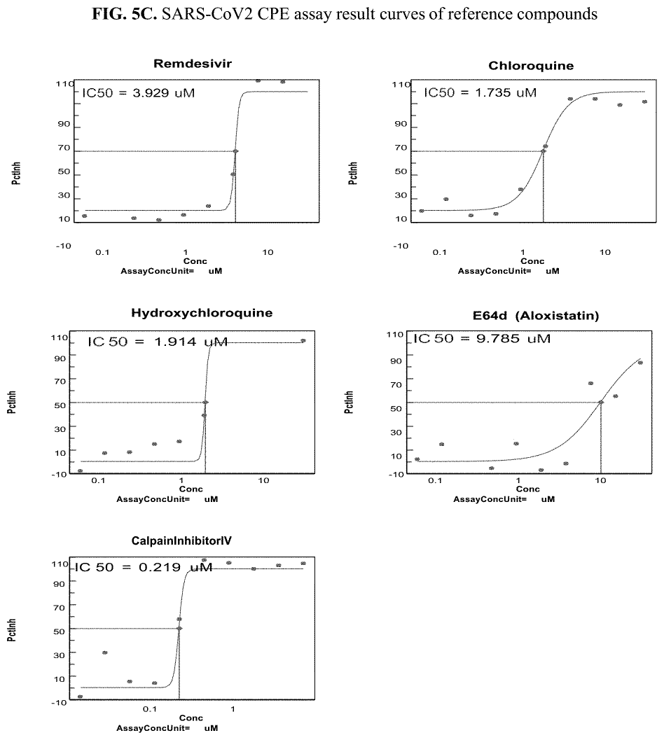

The anti-SARS-CoV-2 activity of IFNT and other reference compounds are shown in FIG. 5.

FIG. 5A presents exemplary data of IFNT-induced inhibition of SARS-CoV-2 in CPE assay. SARS-CoV-2 is a member of Coronaviridae family. The method of this CPE assay is described as follows.

Screening Strategy: We employ a cell-based assay measuring the cytopathic effect (CPE) of the virus infecting Vero E6 host cells. The CPE reduction assay is a popular and widely used assay format to screen for antiviral agents because of its ease of use in high throughput screening (HTS). (Maddox, et al. 2008 J. Assoc. Lab. Automation 2008; 13:168-73; Severson, et al. 2007 J Biomol Screen 12(1):33-40.) In this assay, host cells infected with virus die as a consequence of the viral infection and a simple and robust cell viability assay is the readout. The CPE reduction assay indirectly monitors the effect of antiviral agents acting through various molecular mechanisms by measuring the viability of host cells three days after inoculation with virus. Antiviral compounds are identified as those that protect the host cells from the cytopathic effect of the virus, thereby increasing viability.

Preparation of Assay Ready Plates: Compound stock solution supplied as 0.7 mg/ml in PBS was transferred into an Echo® Qualified 384-Well Polypropylene Source Microplate (Labcyte P-05525). The compound was serially diluted 3-fold in PBS nine times. Using a Labcyte ECHO 550 acoustic liquid handling system a 127.5 nL aliquot of each diluted sample was dispensed into wells of a Corning 3764BC assay plate. This resulted in a 235-fold dilution of each sample in a final assay volume of 30 μL to give the following final concentrations (μg/ml) in the assay:

| | | | | | | | | | |
|---|---|---|---|---|---|---|---|---|---|
| 3.0 | 1.0 | 0.333 | 0.111 | 0.0370 | 0.0123 | 0.00412 | 0.00137 | 0.00046 | 0.00015 |

Method for measuring antiviral effect of compounds: Vero E6 cells selected for expression of the SARS CoV receptor (ACE2; angiotensin-converting enzyme 2) were used for the CPE assay. (Severson, et al. 2007 J Biomol Screen 12(1):33-40.) Cells were grown in MEM/10% HI FBS and harvested in MEM/1% PSG supplemented 2% HI FBS. Cells were batch inoculated with SARS CoV-2 (USA_WA1/2020) at M.O.I. ˜0.002 which results in ˜5% cell viability 72 hours post infection. A 5 ul aliquot of assay media was dispensed to all wells of the assay plates, then the plates were transported into the BSL-3. In the BSL-3 facility a 25 μL aliquot of virus inoculated cells (4000 Vero E6 cells/well) was added to each well in columns 3-24. The wells in columns 23-24 contain virus infected cells only (no compound treatment). A 25 μL aliquot of uninfected cells was added to columns 1-2 of the assay plates for the cell only (no virus) controls. After incubating plates at 37° C./5% CO₂ and 90% humidity for 72 hours, 30 μL of Cell Titer-Glo (Promega) was added to each well. Luminescence was read using a BMG CLARIOstar plate reader following incubation at room temperature for 10 minutes to measure cell viability. Raw data from each test well was normalized to the average signal of non-infected cells (Avg Cells; 100% inhibition) and virus infected cells only (Avg Virus; 0% inhibition) to calculate % inhibition of CPE using the following formula: % inhibition CPE=100*(Test Cmpd−Avg Virus)/(Avg Cells−Avg Virus). Plates were sealed with a clear cover and surface decontaminated prior to luminescence reading.

Method for measuring cytotoxic effect of compounds: Compound cytotoxicity was assessed in a BSL-2 counter screen as follows: Host cells in media were added in 25 μl aliquots (4000 cells/well) to each well of assay plates prepared with test compound as above. Cells only (100% viability) and cells treated with hyamine at 100 μM final concentration (0% viability) served as the high and low signal controls, respectively, for cytotoxic effect in the assay. After incubating plates at 37° C./5% CO2 and 90% humidity for 72 hours, plates were brought to room temperature and 30 μl Cell Titer-Glo (Promega) was added to each well. Luminescence was read using a BMG PHERAstar plate reader following incubation at room temperature for 10 minutes to measure cell viability.

Result: IFNT potently inhibits SARS-CoV-2 in CPE assay with IC50=2.1 nM, which is 1857 times more potent than Remdesivir (IC50=3.9 μM), and 910 times more potent than Hydroxychloroquine (IC50=1.91 μM) in the same assay.

FIG. 5B presents exemplary cell viability data of IFNT. Cytotoxicity evaluation was conducted in parallel with CPE assay. Cytotoxic effect of IFNT was also tested on host Vero E6 cells at the same ten concentrations used for the anti-viral assay in parallel. Cell viability was measured using Promega Cell Titer Glo. CC₅₀ values were calculated from a four-parameter logistic fit of the data.

FIG. 5C shows exemplary data of remdesivir, chloroquine, hydroxychloroquine, aloxistatin, Calpain Inhibitor IV in the CPE assay. The assays were performed as in FIG. 5A.

Table 5 shows exemplary data of the anti-SARS-CoV-2 CPE assay.

### Example 6

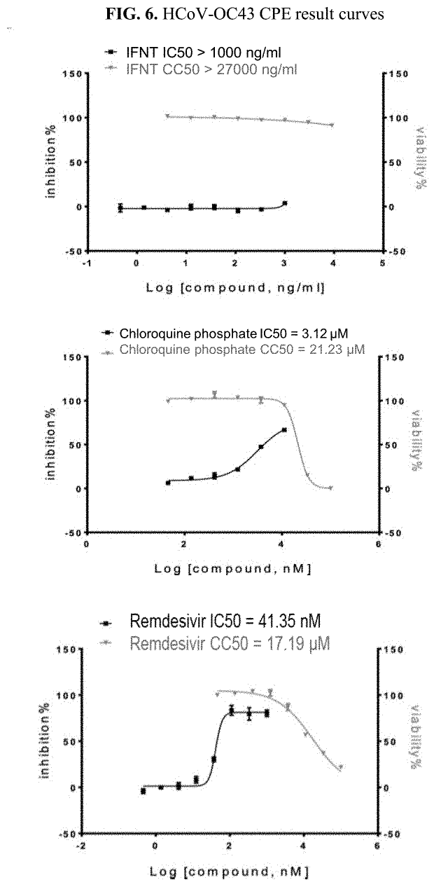

FIG. 6 presents exemplary data of IFNT-induced inhibition of hCoV-OC43 in CPE assay. Remdesivir and chloroquine phosphate were used as reference compounds. The method of this CPE assay is as follows. Test samples and reference compounds were assayed at 8 concentrations with 3-fold dilutions starting at 1000 ng/mL in duplicates. In 96-well plates, Huh7 cells were seeded at an appropriate density and cultured at 37° C. and 5% CO₂ for 4-6 hours. Test samples were added into wells and the plates were incubated at 37° C. and 5% CO₂ for 24 hours. Then medium in each well were replenished with medium containing serially diluted samples/reference compounds and virus (300 TCID50 hCoV-OC43 vs 8000 Huh7 cells).

The resulting cultures were kept under the same conditions for additional 7 days until virus infection in the virus control displays significant CPE. Cytotoxicity of the compounds were assessed under the same conditions, but without virus infection, in parallel. Test samples and reference compounds were assayed at 8 concentrations with 3-fold dilutions starting at 27,000 ng/mL in duplicates. Cell viability was measured by CellTiter Glo following the manufacturer's manual. IC₅₀ and CC₅₀ values were calculated with GraphPad Prism software.

IFNT did not show any anti-viral effect for OC43. This shows its anti-viral selectivity.

TABLE 6 shows exemplary result of anti-OC43 activity of IFNT.

TABLE 1
ZIKV Plaque Reduction Assay

| Results | | | Inhibition (%) (IU/ml) | | | | |
|---|---|---|---|---|---|---|---|
| Sample ID | EC₅₀ | Unit | 100 | 20 | 4 | 0.8 | 0.16 |
| IFN-β | 4.66 | IU/mL | 100 | 100 | 33.93 | 21.43 | 21.43 |
| Results | | | Inhibition (%) (ng/mL) | | | | |
| Sample ID | EC₅₀ | Unit | 200 | 120 | 80 | 40 | 20 |
| IFNT* | <20 | ng/mL | 100 | 100 | 100 | 73.53 | 58.82 |

*IFNT was incubated with cells overnight pre-infection.

TABLE 2
Anti ZIKV (ZIKA/PRVAB59) CPE Assay Result

| Results (ng/ml) | | | Average Activity(%) (ng/ml) | | | | | | | |
|---|---|---|---|---|---|---|---|---|---|---|
| Sample ID | EC₅₀ | CC₅₀ | 300 | 100 | 33.33 | 11.11 | 3.70 | 1.23 | 0.41 | 0.14 |
| IFNT* | 27.80 | >300 | 102.22 | 83.43 | 55.12 | 33.90 | 17.72 | 13.87 | 3.78 | 8.94 |
| (Overnight pre- | | | | | | | | | | |
| incubation) | | | | | | | | | | |
| IFNT* (1 hr pre- | 27.90 | >300 | 94.50 | 74.36 | 58.50 | 41.78 | 31.14 | 22.40 | 18.81 | 12.12 |
| incubation) | | | | | | | | | | |

| Results (IU/ml) | | | Average Activity(%) (IU/ml) | | | | | | | |
|---|---|---|---|---|---|---|---|---|---|---|
| Sample ID | EC₅₀ | CC₅₀ | 2000 | 400 | 80 | 16 | 3.2 | 0.64 | 0.13 | 0.03 |
| IFN-beta | 5.39 | >2000 | 101.94 | 106.29 | 106.71 | 86.73 | 27.59 | 6.46 | −6.80 | 1.85 |

*IFNT was incubated with cells overnight or 1 hr pre-infection.

TABLE 3
Dengue Virus Plaque Reduction Assay

| Results | |
|---|---|
| Sample | Inhibition (%) (ng/ml) |

| ID | EC₅₀ | Unit | 600.00 | 200.00 | 66.67 | 22.22 | 7.41 | 2.47 | 0.82 | 0.27 | 0.09 | 0.03 |
|---|---|---|---|---|---|---|---|---|---|---|---|---|
| IFNT* | 1.09 | ng/ml | 100.00 | 100.00 | 100.00 | 100.00 | 98.59 | 80.28 | 43.66 | 21.13 | 4.23 | 12.68 |

| Results | | | | | | | | | | | | |
|---|---|---|---|---|---|---|---|---|---|---|---|---|
| Sample | | | Inhibition (%) (μM) | | | | | | | | | |
| ID | EC₅₀ | Unit | 300.000 | 100.000 | 33.333 | 11.111 | 3.704 | 1.235 | 0.412 | 0.137 | 0.046 | 0.015 |
| Ribavirin | 32.20 | μM | 100.00 | 98.65 | 60.81 | 24.32 | 20.27 | 31.08 | 13.51 | 10.81 | 14.86 | 14.86 |

*IFNT was incubated with cells overnight pre-infection.

TABLE 4
JEV and YFV Plaque Reduction Assay

| Test | CC50 | EC50 for JEV | EC50 for YFV |
|---|---|---|---|
| compound ID | (ng/mL) | (ng/mL) | (ng/mL) |
| IFNT | >600 | 4.6 | 69.05 |

TABLE 5
SARS-CoV-2 CPE assay data

| Result | | | | | | | | | | | | |
|---|---|---|---|---|---|---|---|---|---|---|---|---|
| CPD | IC₅₀ | CC₅₀ | Average Inhibition (%) (ng/ml) | | | | | | | | | |
| ID | (ng/ml) | (ng/ml) | 3000 | 1000 | 330 | 110 | 37 | 12 | 4 | 1 | 0.5 | 0.2 |
| IFNT | 42 | >3000 | 82.35 | 92.81 | 92.33 | 95.22 | 37.72 | −7.67 | −5.97 | 3.2 | −0.99 | 0.61 |

TABLE 6
HCoV OC43 CPE assay data

| Results (ng/ml) | | | | | | | | | | | |
|---|---|---|---|---|---|---|---|---|---|---|---|
| CPD | CPD | | | Average Inhibition (%) (ng/ml) | | | | | | | |
| No. | ID | EC₅₀ | CC₅₀ | 1000.00 | 333.33 | 111.11 | 37.04 | 12.35 | 4.12 | 1.37 | 0.46 |
| 1 | IFNT | >1000 | >27000 | 3.83 | −3.25 | −4.47 | −0.62 | −0.58 | −3.92 | −1.21 | −1.63 |

| Results (ng/ml) | | | | | | | | | | | |
|---|---|---|---|---|---|---|---|---|---|---|---|
| CPD | CPD | | | Average Viability (%) (ng/ml) | | | | | | | |
| No. | ID | EC₅₀ | CC₅₀ | 27000.00 | 9000.00 | 3000.00 | 1000.00 | 333.33 | 111.11 | 37.04 | 12.35 |
| 1 | IFNT | >1000 | >27000 | 91.17 | 94.73 | 97.25 | 97.25 | 98.96 | 100.57 | 99.44 | 101.51 |

| Results | | | | | | | | | | | |
|---|---|---|---|---|---|---|---|---|---|---|---|
| CPD | | EC₅₀ | CC₅₀ | Average Inhibition (%) (nM) | | | | | | | |
| No. | CPD ID | (nM) | (μM) | 1000.00 | 333.33 | 111.11 | 37.04 | 12.35 | 4.115 | 1.372 | 0.457 |
| 2 | Remdesivir | 41.35 | 17.19 | 80.35 | 79.54 | 83.44 | 30.40 | 8.25 | 1.54 | −0.10 | −4.12 |

| Results | | | | | | | | | | | |
|---|---|---|---|---|---|---|---|---|---|---|---|
| CPD | | EC₅₀ | CC₅₀ | Average Viability (%) (μM) | | | | | | | |
| No. | CPD ID | (nM) | (μM) | 100.00 | 33.33 | 11.11 | 3.704 | 1.235 | 0.412 | 0.137 | 0.046 |
| 2 | Remdesivir | 41.35 | 17.19 | 21.77 | 36.88 | 56.84 | 86.91 | 102.28 | 104.10 | 101.58 | 99.99 |

| Results | | | | | | | | | | | |
|---|---|---|---|---|---|---|---|---|---|---|---|
| CPD | | EC₅₀ | CC₅₀ | Average Inhibition (%) (μM) | | | | | | | |
| No. | CPD ID | (μM) | (μM) | 100.00 | 33.33 | 11.11 | 3.70 | 1.23 | 0.412 | 0.137 | 0.046 |
| 3 | chloroquine | 3.12 | 21.23 | −84.89 | −43.28 | 66.65 | 47.44 | 21.61 | 14.41 | 11.72 | 6.25 |
| | phosphate | | | | | | | | | | |

| Results | | | | | | | | | | | |
|---|---|---|---|---|---|---|---|---|---|---|---|
| CPD | | EC₅₀ | CC₅₀ | Average Viability (%) (μM) | | | | | | | |
| No. | CPD ID | (μM) | (μM) | 100.00 | 33.33 | 11.11 | 3.704 | 1.235 | 0.412 | 0.137 | 0.046 |
| 3 | chloroquine | 3.12 | 21.23 | 0.09 | 14.93 | 94.86 | 100.63 | 103.48 | 106.75 | 101.83 | 98.86 |
| | phosphate | | | | | | | | | | |

### Example 7

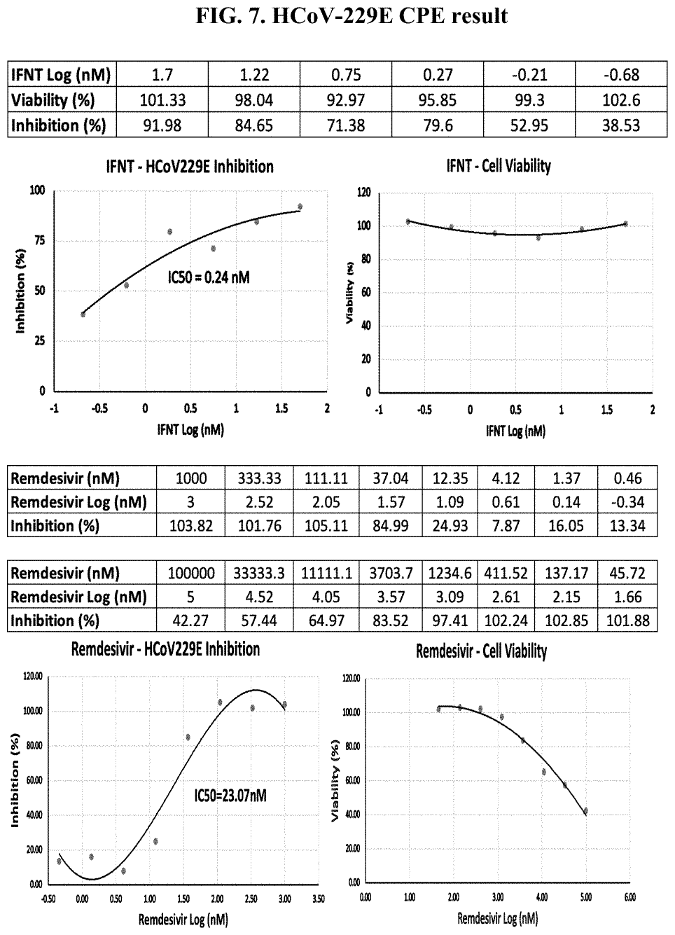

FIG. 7 shows exemplary data of IFNT inhibited hCoV-229E in CPE assay. The method of this CPE assay is as follows: In 96-well plates, MRCS cells were seeded at an appropriate density and cultured at 37° C. and 5% CO₂ overnight. Test samples were added into wells and the plates were incubated (200 TCID 50 hCoV-229E vs 20,000 MRCS cells) at 37° C. and 5% CO₂ for 2 hours. Then medium in each well was replenished with medium containing serially diluted samples and virus. The resulting cultures were kept under the same conditions for additional 3 days until virus infection in the virus control displayed significant CPE. Cytotoxicity of the compounds was assessed under the same conditions, but without virus infection, in parallel. Cell viability was measured by CellTiter Glo following the manufacturer's manual. IC₅₀ and CC₅₀ values were calculated with GraphPad Prism software.

FIG. 7 also shows exemplary data of CPE and the cell viability data of remdesivir. The assays were performed as IFNT treatment. FIG. 7 shows exemplary data of IFNT potently inhibited the hCoV229E with an IC50 of 0.241 nM, which is 100× more potent than remdesivir's IC50 of 23.07 nM, and 10,000× more potent than Chloroquine (IC50=2.2 μM) (data not shown), the two currently FDA-approved anti-Covid-19 drugs. The IC50s of Ribavirin is 30 μM in this assay (data not shown).

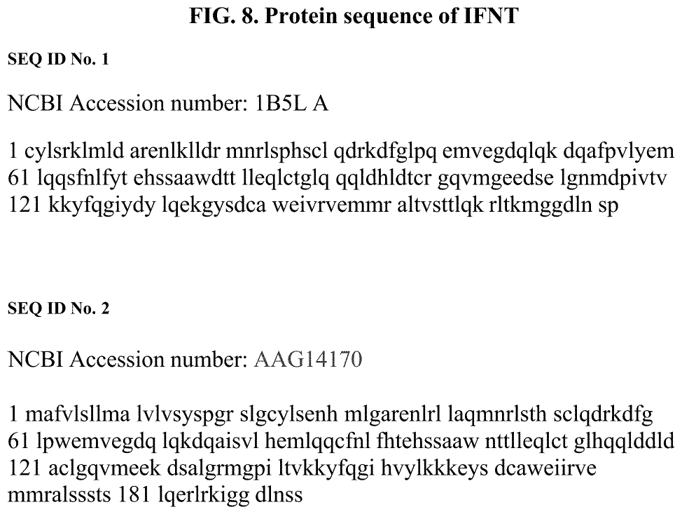

FIG. 8 shows the two protein sequences of IFNT.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term “consisting essentially of” does not exclude pharmacologically inactive or inert agents, e.g., pharmaceutically acceptable excipients, carriers or diluents. The term “consisting of” shall mean excluding trace elements of other ingredients and substantial method steps. Embodiments defined by each of these transition terms are within the scope of this invention.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### Incorporation by Reference

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### Equivalents

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for treating a viral infection, or a related disease or condition, comprising administering to a subject in need thereof a therapeutically effective amount of interferon tau (IFNT) and a pharmaceutically acceptable excipient, carrier, or diluent, wherein the viral infection comprises infections of flavivirus and/or coronavirus.

2. The method of claim 1, wherein the viral infection comprises infection of a flavivirus or coronavirus.

3. The method of claim 1, wherein the viral infection comprises infection of one or more of ZIKA, DENV, YFV, JEV, West Nile and Powassan viruses.

4. The method of claim 1, wherein the viral infection comprises infection of a coronavirus.

5. The method of claim 1, wherein the viral infection comprises infection of one or more of hCoV-229E, SARS-related coronaviruses and MERS-related coronaviruses.

6. The method of claim 1, wherein the viral infection comprises infection of SARS-CoV-2.

7. (canceled)

8. The method of claim 1, wherein the related disease or condition is pneumonia, acute respiratory distress syndrome (ARDS), inflammatory disorder, a cardiovascular disorder, common cold, or flu.

9-12. (canceled)

13. The method of claim 1, wherein the IFNT comprises a mammalian IFNT.

14. The method of claim 1, wherein the IFNT comprises non-human mammalian IFNT.

15. The method of claim 1, wherein the IFNT comprises recombinant IFNT.

16. The method of claim 1, wherein the IFNT comprises an amino acid sequence that is at least 70% homologous with SEQ ID No. 1 or SEQ ID No. 2.

17. (canceled)

18. The method of claim 1, wherein the IFNT comprises an amino acid sequence set forth in SEQ ID NO. 1 or SEQ ID No. 2.

19. (canceled)

20. The method of claim 1, wherein the administration comprises oral administration.

21. The method of claim 1, wherein the administration comprises intravenous, intramuscular, subcutaneous, and/or inhaled administrations.

22. The method of claim 1, further comprising administering a second therapeutic agent.

23. The method of claim 22, wherein the second therapeutic agent is an antiviral agent.

24-31. (canceled)

32. The method of claim 23, wherein the second therapeutic agent is administered prior to, concomitant with or after the administration of IFNT.

33. A method for inhibiting viral replication in cells, comprising administering to a subject in need thereof an amount of interferon tau (IFNT) effective to inhibit viral replication in the cells, wherein the inhibited virus is selected from flavivirus or coronavirus, and a pharmaceutically acceptable excipient, carrier, or diluent.

34-37. (canceled)

38. The method of claim 1, wherein IFNT is administered at a dosage in the range from about 0.1 mg to about 100 mg per day.

39. (canceled)

40. A pharmaceutical composition comprising interferon tau (IFNT) and a pharmaceutically acceptable excipient, carrier, or diluent.

41-66. (canceled)
